(19) United States
(12) Patent Application Publication
(10) Pub. No.: US 2008/0299543 A1
(43) Pub. Date: Dec. 4, 2008

(54) Method for Assessing Trace Element Related Disorders in Blood Plasma

(75) Inventor: Juergen Gailer, Calgary (CA)

Correspondence Address: FULBRIGHT & JAWORSKI L.L.P., 600 CONGRESS AVE., SUITE 2400, AUSTIN, TX, 78701, US

(73) Assignee: UTI LIMITED PARTNERSHIP

(21) Appl. No.: 12/129,233

(22) Filed: May 29, 2008

Related U.S. Application Data
(60) Provisional application No. 60/941,212, filed on May 31, 2007.

(51) Int. Cl. C12Q 1/00

(52) U.S. Cl. 435/4

(57) ABSTRACT

The present invention provides for spectrometric methods of analyzing plasma or serum for metal distribution in metalloproteins. The methods can be used to assess such conditions as toxicity and disease in subjects.

### BACKGROUND OF THE INVENTION

A. Field of the Invention

The present invention relates generally to the field of spectrophotometric analysis of plasma or serum samples, and more particularly, to the determination of metal species contained in plasma or serum.

B. Related Art

It has long been known that blood serum may be spectrophotometrically analyzed by combining a serum sample with one or more selected reagents which will combine with a selected component within that sample to form a colored entity. Upon a subsequent spectrophotometric analysis of that sample, the concentration of that component within that sample may be determined. It also has been suggested that multiple component end point determinations may be made within a single reaction medium. For example, in Chem Abstracts 88 (1978), it is suggested that a reagent be composed of two or more compounds may be reacted with two or more of the components of a test solution to give two colored products. The analysis procedure may be simplified if the reagent also includes all auxiliary compounds used in analysis, as for example, buffers, masking agents, etc. The optimization scheme disclosed in this abstract includes the selection of preferred conditions of analysis; that is, preferred calorimetric reagent compositions and preferred wavelengths suited for use during a certain multi-component spectrophotometric analysis. In particular, this abstract discloses a reagent composed of murexide, calmagite, and other materials for the detection of both calcium and magnesium in a given serum sample.

It has also been proposed to make kinetic determinations of the enzymatic activities exhibited by a plurality of enzymes contained in a single aqueous reaction medium. In accordance with this proposed method, known quantities of substrates, one of which is “consumed” by each of the enzymes to be determined, and any reagents required for the measurement of substrate or reaction product concentrations at preselected wavelengths may be added to the reaction medium and as employed permit enzymatic reactions to proceed simultaneously under the same reaction conditions. By sequentially measuring changes in the absorbance or fluorescence of the reaction medium over time at said wavelengths, the concentration of a corresponding number of enzymes may be determined by formulating simultaneous equations of the first degree. See U.S. Pat. Nos. 3,925,162 and 3,718,433.

For other papers and disclosures relating to spectrophotometric analysis of various serum components, please refer to West German Auslegeschrift 2558536 (Offenlegunstag, Jul. 7, 1977); Luderer (1975); Banauch et al. (1975); Kageyama (1971). See also, U.S. Pat. Nos. 3,907,645; 3,703,591; 3,925,164; 4,102,646; 3,899,297 and Sterns, (1969).

While considerable progress in the determination of blood serum components has been made, various practical considerations have somewhat limited the success of prior art methods. Ideally, simple, low cost reagents or reagent sets exhibiting extended shelf life are needed to cover a wide range of serum components. Such reagents or reagent sets preferably should be suitable for use with samples maintained within normal temperature ranges to produce reaction media which are readily analyzed to provide statistically significant determinations. Often, due to the differing reaction kinetics of the component specific calorimetric reactions, analysis of multiple components in a single reaction medium may require numerous, sequential photometric determinations, first for one component, and then, substantially later for a second component.

U.S. Pat. No. 4,425,427 discloses method, kits and reagents for the simultaneous, kinetic spectrophotometric analysis of blood serum samples for multiple components. Pairs of components which may be simultaneously analyzed are cholesterol and triglyceride; glucose and urea; uric acid and gamma glutamyl transferase; calcium and magnesium; albumins and total protein. A more particular aspect of examining plasma content involves assessing metal distribution, for example, in the context of metal poisoning. U.S. Pat. No. 6,248,592 describes methods for measuring lead concentrations in blood including the use of resonant laser ablation to analyze samples of blood for lead content. The sample is placed on a lead-free, electrically conducting substrate and irradiated with a single, focused laser beam which simultaneously vaporizes, atomizes, and resonantly ionizes an analyte of interest in a sample. The ions are then sorted, collected and detected using a mass spectrometer.

### SUMMARY OF THE INVENTION

The present invention provides for analytical methods for the direct analysis of human blood plasma or serum (in as little as 0.5 ml) for copper, iron and zinc containing metalloproteins and metallopeptides (metals bound to small molecular weight compounds; see Table 1). Crude size exclusion chromatographic separation of the plasma proteins into bands is used in conjunction with a multi-element-specific detector (inductively coupled plasma atomic emission spectrometer) to simultaneously detect the separated metalloproteins and metallopeptides in an on-line fashion to obtain the plasma “metalloproteome.” The developed methodology can be used to diagnose several known trace element related disorders which are associated with increased or decreased concentrations of certain plasma metalloproteins and/or metallopeptides (or their presence or absence) within as short as about 24 minutes. In addition, this methodology can be used to study the effect of compounds that are added to plasma (or blood) on the metalloproteome as a proxy of the toxicity.

Thus, in accordance with the present invention, there is provided a method of measuring metal distribution in plasma or serum comprising (a) providing a plasma or serum sample; (b) subjecting said plasma or serum sample to size exclusion chromatography (SEC) to obtain SEC effluent comprising separated plasma or serum proteins; (c) feeding SEC effluent obtained in step (b) directly into an inductively-coupled plasma atomic emission spectrometer (ICP-AES) to determine the metal content thereof; and (d) associating the metal content determined in step (c) with plasma or serum proteins separated in step (b). The time from step (b) to step (d) may be less than 30 minutes and as low as about 24 minutes. The following metals may be simultaneously detected: Cu, Zn, and Fe. The AES may be inductively coupled plasma AES. The plasma or serum may be from rabbit, dog, cat, rat, mouse, sheep, goat, cow, pig or horse, or from a human. The human plasma or serum may be obtained from a subject that is suspected of having a condition that effects the metalloprotein content of blood plasma or serum. The condition may more specifically be hemochromatosis, Wilson's Disease, metal poisoning, infection or other essential trace element imbalance-related disorders. The method may further comprise the step of obtaining said blood sample from a subject and preparing said plasma or serum sample therefrom. The amount of the plasma or serum sample subjected to SEC may be 500 μl. Step (d) may comprise computer-assisted processing of data from said SEC-ICP-AES. The metal content of said human subject may be assessed from at least two different time points. The plasma or serum sample should be essentially free of red blood cells and hemoglobin (from lysed red blood cells), i.e., undetectable or trace amounts.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Schematic depiction of the instrumental analytical SEC-ICP-AES setup.

FIG. 2—Simultaneous multielement-specific chromatograms of rabbit plasma. Superdex 200 10/300 GL (13 μm particle size) SEC column with a phosphate buffered saline mobile phase (pH 7.4, 22° C.); flow rate: 1.0 ml/min; injection volume: 500 μl; detector: ICP-AES. Emission lines for Cu @ 324.754 nm (green), Fe @ 259.940 nm (blue), and Zn @ 213.856 nm (red). The positive identification of the metalloproteins α2-macroglobulin, ceruloplasmin, ferritin and transferrin in collected fractions by various enzyme-based assays is indicated by horizontal bars.

FIG. 3—Simultaneous Cu. Fe and Zn-specific chromatograms of rabbit plasma collected over a two hour time period. On a Superdex 200 10/300 GL (13 μm particle size) SEC column with a phosphate buffered saline mobile phase (pH 7.4, 22° C.); flow rate: 1.0 ml/min; injection volume 500 μl; detector: ICP-AES. Cu-, Fe- and Zn-specific chromatograms were obtained in 0.5 h intervals at room temperature and the emission lines of each element [Cu @ 324.754 nm, Fe @ 259.940 nm and Zn @ 213.856 nm] were plotted on top of each other.

FIG. 4—Simultaneous multi-element-specific chromatograms of plasma from a healthy human. Superdex 200 10/300 GL (13 μm particle size) SEC column with a phosphate buffered saline mobile phase (pH 7.4, 22° C.); flow rate: 1.0 ml/min; injection volume: 500 μl; detector: ICP-AES. Emission lines for Cu @ 324.754 nm (green), Fe @ 259.940 nm (blue), and Zn @ 213.856 nm (red).

FIG. 5—Simultaneous Cu-, Fe- and Zn-specific chromatograms of human plasma collected over a two hour time period. On a Superdex 200 10/300 GL (13 μm particle size) SEC column with a phosphate buffered saline mobile phase (pH 7.4, 22° C.); flow rate: 1.0 ml/min; injection volume 500 μl; detector: ICP-AES. Cu-, Fe- and Zn-specific chromatograms were obtained in 0.5 h intervals at room temperature and the emission lines of each element [Cu @ 324.754 nm, Fe @ 259.940 nm and Zn @ 213.856 nm] were plotted on top of each other.

### DETAILED DESCRIPTION OF THE INVENTION

### I. The Present Invention

The present invention represents an improved analytical method for the direct analysis of mammalian blood plasma or serum for metalloproteins and metallopeptides. Plasma, the liquid component of blood, separated from red blood cells by centrifugation, is fed through a size exclusion chromatography (SEC) column to separate the proteins into crude bands. An inductively coupled plasma atomic emission spectrometer (ICP-AES) is used as an on-line multielement-specific detector to simultaneously detect levels of essential trace elements that are inherently associated with metalloproteins and metallopeptides. The data can be used to diagnose early and advanced stage human diseases that result from the excess or deficiency of individual metalloproteins and metallopeptides.

A major innovation in the present invention is the ability to simultaneously measure multiple metalloproteins and metallopeptides of more than one element in a rapid, cost-effective fashion by direct transfer of the SEC effluent into the ICP-AES to obtain results within about 24 minutes. This methodology enables the diagnosis of multiple human diseases from one analysis which is superior to the many methods which exist to measure individual metalloproteins.

### II. Metalloproteins

In biochemistry, a metalloprotein is a generic term for a protein that contains a metal cofactor. The metal may be an isolated ion or may be coordinated with a nonprotein organic compound, such as the porphyrin found in hemoproteins. In some cases, the metal is co-coordinated with a side chain of the protein and an inorganic nonmetallic entity. This kind of protein-metal-nonmetal structure is seen in iron-sulfur clusters. Table 1 provides a list of metalloproteins and metallopeptides in human plasma and serum, as we as concentrations and amounts of metal ions.

A. Copper

Type I copper centers (T1Cu) are characterized by a single copper atom coordinated by two histidine residues and a cysteine residue in a trigonal planar structure, and a variable axial ligand. In class I T1Cu proteins (e.g., amicyanin, plastocyanin and pseudoazurin) the axial ligand is a methionine, whereas aminoacids other than methionine (e.g., glutamine) give rise to class II T1Cu copper proteins. Azurins contain the third type of T1Cu centres: besides a methionine in one axial position, they contain a second axial ligand (a carbonyl group of a glycine residue). T1Cu-containing proteins are usually called “cupredoxins,” and show similar three-dimensional structures, relatively high reduction potentials (>250 mV), and strong absorption near 600 nm (due to S→Cu charge transfer), which usually gives rise to a blue color. Cupredoxins are therefore often called “blue copper proteins.” This may be misleading, since some T1Cu centres also absorb around 460 nm and are therefore green. When studied by EPR spectroscopy, T1Cu centres show small hyperfine splittings in the parallel region of the spectrum (compared to common copper coordination compounds).

Type II copper centres (T2Cu) exhibit a square planar coordination by N or N/O ligands and an axial EPR spectrum with copper hyperfine splitting in the parallel region similar to that observed in regular copper coordination compounds. Since no sulphur ligation is present, the optical spectra of these centres lack distinctive features. T2Cu centres occur in enzymes, where they assist in oxidations or oxygenations.

Type III copper centres (T3Cu) are binuclear centres consisting of two copper atoms, each coordinated by three histidine residues. These proteins exhibit no EPR signal due to strong antiferromagnetic coupling (i.e., spin pairing) between the two S=½ metal ions due to their covalent overlap with a bridging ligand. These centres are present in some oxidases and oxygen-transporting proteins (e.g., hemocyanin and tyrosinase).

Binuclear Copper A centres (Cu_{A}) are found in cytochrome c oxidase and nitrous-oxide reductase (EC 1.7.99.6). The two copper atoms are coordinated by two histidines, one methionine, a protein backbone carbonyl oxygen, and two bridging cysteine residues.

Copper B centres (Cu_{B}) are found in cytochrome c oxidase. The copper atom is coordinated by three histidines in trigonal pyramidal geometry.

Tetranuclear Copper Z centre (Cu_{Z}) is found in nitrous-oxide reductase. The four copper atoms are coordinated by seven histidine residues and bridged by a sulfur atom.

B. Iron

A hemoprotein (also haemoprotein), or heme protein, is a metalloprotein containing a heme prosthetic group, either covalently or noncovalently bound to the protein itself. The iron in the heme is capable of undergoing oxidation and reduction (usually to +2 and +3, though stabilized ferryl [Fe⁺⁴] compounds are well known in the peroxidases). Hemoproteins have diverse biological functions including transport (hemoglobin, myoglobin, neuroglobin, cytoglobin, leghemoglobin), catalysis (peroxidases, cytochrome c oxidase, ligninases), active membrane transport (cytochromes, electron transfer, cytochrome c) and sensory (FixL—oxygen sensor; sGC—nitric oxide sensor).

C. Zinc

Zinc is found in relatively low abundance in nature, e.g., nominally 70 ppm in the earth's crust and approximately 0.01 ppm in sea water. Yet, zinc plays an essential role in biology in the form of zinc metalloproteins and as a regulatory agent in homeostasis. In zinc metalloproteins, zinc can play a structural role or a catalytic one. The propensity for Zn²⁺ to occupy tetrahedral sites and less commonly octahedral or pentacoor-dinated sites in metalloproteins facilitates the structurally based functions, while more than 300 catalytically active zinc metalloproteins are known.

TABLE 1
MOLECULAR PROPERTIES AND RELATIVE ABUNDANCES
OF THE MAJOR METALLOPROTEINS AND METALLOPEPTIDES
IN HUMAN PLASMA AND SERUM

| | Metalloprotein | | | plasma or | | |
|---|---|---|---|---|---|---|
| | or entity that | | # of metal | serum | | |
| | contains bound | | bound per | protein | | |
| Metal | metal | kDa | protein | conc. | | Ref. |
| Fe | ferritin | 450 | ≦4500 | 10-250 | μg/L | |
| | transferrin | 79.7 | 2 | 1.8-3.7 | μg/L | |
| Cu | blood coag. | 330 | 1 | ~10 | mg/L | |
| | Factor V | | | | | |
| | transcuprein | 270 | 0.5 | ~180 | μg/L* | |
| | ceruloplasmin | 132 | 6 | 0.2-0.6 | g/L | |
| | albumin | 66 | 1 | 36.1-53.6 | g/L | |
| | EC-SOD | 165 | 4 | — | | |
| | Cu/Zn-SOD | 31 | — | — | | |
| | peptides & AA | <5 | — | — | | — |
| Zn | α₂ macroglobulin | 725 | 5 | 1.1-3.7 | g/L | |
| | albumin | 66 | 1 | 36.1-53.6 | g/L | — |

| | | | |
|---|---|---|---|
| EC-SOD | 165 | 4 | — |
| Cu/Zn-SOD | 31 | — | — |

*rat plasma

### III. Size Exclusion Chromatography

Size exclusion chromatography (SEC) is a chromatographic method in which molecules are separated based on their size, or in more technical terms, their hydrodynamic radius. It is usually applied to separate large molecules or macromolecular complexes such as proteins and industrial polymers. When an aqueous solution is used to transport the sample through the column, the technique is known as gel filtration chromatography. The main application of gel filtration chromatography is the fractionation of proteins and other water-soluble polymers. This technique should not be confused with gel electrophoresis, where an electric field is used to “pull” or “push” molecules through the gel depending on their electrical charges.

SEC is a widely used technique for the purification and analysis of synthetic and biological polymers, such as proteins, polysaccharides and nucleic acids. Biologists and biochemists typically use a gel medium, usually polyacrylamide, dextran or agarose to analyze aqueous samples at low backpressure. Polymer chemists typically use either a silica or crosslinked polystyrene medium under a higher backpressure. These media are also referred to as the stationary phase.

The advantage of this method is that the various solutions can be applied, while preserving the biological activity of the molecules to be separated. The technique is generally combined with other separation techniques which further separate molecules by other characteristics, such as acidity, basicity, charge, and affinity for certain compounds.

The underlying principle of SEC is that molecules of different sizes will elute from a stationary phase at different times. This results in the separation of molecules (contained in a liquid sample) based on their size. Provided that all molecules are loaded simultaneously or near simultaneously, molecules of the same size should elute together.

This is usually achieved with an apparatus called a column, which consists of a hollow tube tightly packed with extremely small porous polymer beads designed to have pores of different sizes. These pores may be depressions on the surface or channels through the bead. As the solution travels down the column, some molecules enter into the pores. Larger molecules cannot enter into as many pores. The larger the molecules, the less overall volume to traverse over the length of the column, and the faster the elution. The sample molecules are carried through the column by the eluent. The void volume consists of any particles too large to enter the pores, and the column volume is known as the inclusion volume.

In real life situations molecules in solution do not have a constant, fixed size, resulting in the probability that a molecule which would otherwise be hampered by a pore may pass right by it. Also, the stationary phase particles are not ideally defined; both particles and pores may vary in size. Elution curves therefore resemble gaussian distributions. The stationary phase may also interact in undesirable ways with a molecule and influence retention times, though great care is taken by column manufacturers to use stationary phases which are inert and minimize this issue.

Like other forms of chromatography, increasing the column length will improve the resolution, and increasing the column diameter will increase the capacity of the column. Proper column packing is important to maximize resolution.

With regard to the general operation, the column effluent can be collected in constant volumes, known as fractions. Alternatively, the effluent can be monitored on-line by an appropriate detector, such as a refractive index (R1), an evaporative light scattering (ELS), an ultraviolet (UV) or an ICP-AES detector. The results from the analysis of the collected fractions or the results from the on-line analysis of the column effluent are used to determine the concentration of certain analyte molecules.

### IV. Atomic Emission Spectrometry

In atomic emission spectrometry, liquid samples are generally aspirated into a flame, where vaporization and atomization of the elements (that are contained in the sample) will take place. At temperatures between 2000 K (flame) and 6000 K (plasma) atoms are also excited to higher energy electronic states and the concentration of atoms in the flame (and therefore in the sample) can be obtained by measuring the emission of characteristic wavelengths of radiation which are given off when atoms return to their energetic ground state. The fundamental characteristic of this process is that each element emits a specific wavelength peculiar to its chemical character. Because of its high sensitivity, its ability to distinguish one element from another in a complex sample, its ability to perform simultaneous multi-element analyses, and the ease with which many samples can be automatically analyzed, atomic emission spectroscopy is an extremely important tool in analytical chemistry. Today, emission is mostly achieved in inductively coupled plasmas, which offer approximately twice the temperature that flames do. The high temperature, stability, and chemically inert environment in the plasma eliminate many interferences encountered with flames. Simultaneous multi-element analysis is routine for inductively coupled plasma atomic emission spectrometry (ICP-AES). In the ICP-AES technique it is most common to select a single wavelength for a given element. The intensity of the light that is emitted is proportional to the concentration of that element in the analyzed sample.

All ICP-AES systems consist of several components, the three main aspects being the sample introduction system, the torch assembly, and the spectrometer. The sample introduction system on the ICP-AES normally consists of a peristaltic pump, tubing, a nebulizer, and a spray chamber. The fluid sample is pumped into the nebulizer via the peristaltic pump. The nebulizer generates an aerosol mist and injects humidified Ar gas into the chamber along with the sample. This mist accumulates in the spray chamber, where the largest mist particles settle out as waste and the finest particles are subsequently swept into the torch assembly. Approximately 1% of the total solution eventually enters the torch as a mist, whereas the remainder is pumped away as waste. The fine aerosol mist containing Ar gas and sample is injected into the plasma (through the torch assembly). The radio frequency-generated and maintained Ar plasma, portions of which are as hot as 6,000-8,000 K, excites the electrons. When the electrons return to the ground state at a certain spatial position in the plasma, they emit energy at the specific wavelengths peculiar to the sample's elemental composition.

Light emitted from the plasma is focused through a lens and passed through an entrance slit into the spectrometer (radial view or axial view configuration). The ICP-AES that is used in the present application is an advanced high dispersion Echelle spectrometer which means that the light is separated into its individual wavelengths by means of an Echelle grating, which is analogous to a prism that refracts visible light into its component colors. The separated wavelengths eventually hit individual pixels of a state-of-the-art, large format, programmable array CID (charge injection device) detector in order to measure the light intensity which is correlated to the concentration of a metal in the plasma and the concentration of the metal in the aspired solution.

Since the detector technology utilized in the Prodigy ICP-AES allows the system operator to simultaneously monitor multiple analytical wavelengths along with their spectral backgrounds and any internal standards of interest, the Prodigy can be used as a true simultaneous multi-element-specific detector when hyphenated to a separation technique. The ability to simultaneously measure peak and background emissions is critical to experiments where time varying signals are involved. The reason for this is that it is the net emission intensity (peak minus background) that is used when relating intensity to analyte concentration and many experiments that involve time resolution, also involve changes in parameters (e.g., solvent composition), which can significantly alter background emission intensity. Without simultaneous peak and background measurement, these changes in background signal can mislead the operator into believing that an analytically significant event has occurred, when in fact it was a simple background shift. This advantage, together with the ability of ICP-AES to handle salt-containing solutions, makes the Prodigy ideally suited for the LC analysis of solutions (including biological fluids, such as blood plasma or serum, bile, etc.) containing metals or metalloids in metalloproteins and metallopeptides (throughout the application all liquid chromatographic separation techniques will be referred to as LC).

### V. Sample Preparation

Blood will be obtained by standard phlebotomy procedures. Immediately following blood draw to obtain plasma, protease inhibitors and/or anticoagulants can be added to the blood sample. The tube should be cooled and within 30 minutes, centrifuged at 2000-3000 RCF at 4° C. for 15 min—not to exceed 10,000 RCF (3000 g). Within 30 minutes of centrifugation, the plasma is transferred in aliquots and placed immediately on ice. The aliquots may be frozen at −30° C. until used. 8.5 mL of blood will yield about 2.5-3.0 mL of plasma.

Serum is prepared in a very similar fashion. Venous blood is collected, followed by mixing of protease inhibitors and coagulant with the blood by inversion. The blood is allowed to clot by standing tubes vertically at room temperature (22° C.) for 60 min. The tubes are placed in wet ice for no longer than 2 hours before centrifuging at 1400-2000 RCF for 10 min at 4° C. Within 30 minutes of centrifugation, the supernatant (serum) is transferred in aliquots and placed immediately on ice. The aliquots may be frozen at −30° C. until used.

### VI. Methodology

### A. SEC-ICP-AES System Configuration

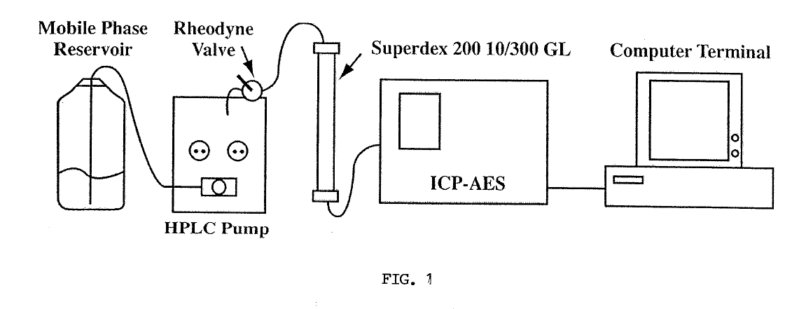

After the equilibration of a prepacked Superdex 200 10/300 GL SEC column (diameter: 10 mm, length: 30 cm) with approximately 60 ml of phosphate buffered saline (PBS)-buffer at a flow rate of 1.0 ml/min (the SEC column exit is not connected to the ICP-AES), the ICP-AES is switched on and the wavelengths for monitoring the elements that will be monitored during plasma analysis are selected: carbon (193.091 nm), copper (324.754 nm), iron (259.940 nm), phosphorus (213.618 nm), sulfur (180.731 nm) and zinc (213.856 nm). After aligning the wavelengths using aqueous solutions of salts containing these elements (concentrations between 10 and 1000 ppm depending on ICP-AES detection limit of the corresponding element), the plasma is positioned by using an aqueous manganese solution (10 ppm). After configuring the ICP-AES for time resolved analysis (TRA mode), the LC column exit is connected to the ICP-AES nebulizer and the SEC-ICP-AES system is now ready for plasma analysis. Before the first plasma analysis, a mixture of two proteins (albumin and lysozyme) is injected in order to establish the performance of the column (to calculate the plate number). A model system is shown in FIG. 1.

### B. Blood Collection and Plasma Preparation

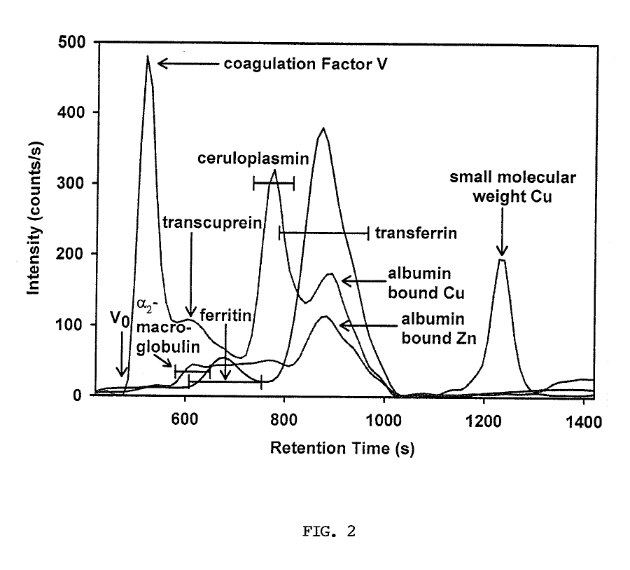

Blood (approximately 7 ml) was collected from male New Zealand white rabbits from the marginal ear vein with 20 gage stainless steel blood collection needles (211 monoject, Sherwood Medical, St. Louis, Mo., USA) into BD Vacutainer tubes (for trace element work, no additive) to each of which 0.7 mg heparin had been added (anticoagulant). After mixing, the blood is centrifuged at 1100 g for 10 min (at 22° C.) to remove all erythrocytes. The clear and yellowish plasma is then collected and injected into a non-steel Rheodyne injection valve (equipped with a 0.5 ml loop) of the LC system. After the injection and a delay of 7 min, data collection was initiated. Data were collected for 1000 s and after the completion of the run the collected data were saved and exported to Sigmaplot for further data processing. A typical chromatogram is shown in FIG. 2.

### VII. Conditions Being Diagnosed

In accordance with the present invention, one can perform diagnostic and prognostic testing on individuals suspected of having, at risk of having or known to have certain diseases. By comparing metalloprotein content of blood/serum with known values for normal and disease states, such diagnoses and prognoses may be accurately made (Table 2 is provided as a guide for such methods). Thus, this information is provided as a general guide.

TABLE 2
NORMAL METALLOPROTEIN CONTENT

| Metalloprotein | Age (yrs.) | Males (g/L) | Females (g/L) |
|---|---|---|---|
| Ceruloplasmin | 0.5-3 | 0.26-0.90 | |
| | 4-12 | 0.25-0.46 | |
| | 13-19 | 0.15-0.50 | |
| | >19 | 0.20-0.60 | |
| Transferrin | 0-1 | 1.40-3.19 | 1.48-3.16 |
| | 2-30 | 1.89-3.58 | 1.80-3.91 |
| | 31-60 | 1.78-3.54 | 1.80-3.72 |
| | >60 | 1.63-3.31 | 2.47-3.66 |
| Ferritin | 0.5-15 | 7-140 (μg/L) | |
| | >15 | 20-250 (μg/L) | 10-120 (μg/L) |

### Ceruloplasmin Levels are Generally Increased in the Following Conditions/Disease States:

Bile duct obstruction
Primary biliary cirrhosis
Hypoplastic anemia
Leukemia
APR (inflammation, infection, surgery, trauma, malignancy)
Rheumatoid arthritis
Physical exercise
Pregnancy (late)
Estrogen therapy

### Ceruloplasmin Levels are Generally Decreased in the Following Conditions/Disease States:

Wilson's disease
Menke's disease
Nephrotic syndrome
Severe liver disease
Primary sclerosing cholangitis
Acute viral hepatitis
Gastroenteropathies
Malnutrition

### Ferritin Levels are Generally Increased in the Following Conditions or Disease States:

Hereditary
Liver disease
Cirrhosis
Hemochromatosis
Acute phase response (infection, surgery, inflammation)
Adult Still's disease
Chronic viral hepatitis
Various neoplastic diseases
Anemia of chronic disease
Chronic renal failiure
Thalassemia
Sideroblastic anemia

### Ferritin Levels are Generally Decreased in the Following Conditions/Disease States:

Iron deficiency
Pregnancy
Chronic blood loss
Frequent blood donations
Existence of colonic polyps

### Transferrin Levels are Generally Increased in the Following Conditions/Disease States:

Iron deficiency
Acute hepatitis
Hypothyroidism
Pregnancy and estrogen therapy

### Transferrin Levels are Generally Decreased in the Following Conditions/Disease States:

Iron overload conditions (e.g. hereditary hemochromatosis)
Acute phase response (inflammation, tissue necrosis, trauma, surgery)
Malignancy
Liver disease
Nephrotic syndrome
Malnutrition
Dialysis patients
Chronic renal failure

The above information is derived from Craig, W. Y., Ledue, T. B., and Ritchie, R. F. (2000). Plasma Proteins: Clinical Utility and Interpretation. Newark, Dade Behring, Inc. Information relating to specific conditions/disease states is provided below.

A. Metal Poisioning

Copper can be present in numerous sources, such as birth control pills, congenital intoxication, copper cookware, copper IUDs, copper pipes, dental alloys, fungicides, ice makers, industrial emissions, insecticides, swimming pools, water (city/well), welding, avocado, beer, bluefish, bone meal, chocolate, corn oil, crabs, gelatin, grains, lamb, liver, lobster, margarine, milk, mushrooms, nuts, organ meats, oysters, perch, seeds, shellfish, soybeans, tofu, wheat germ, and yeast. The effects of copper poisoning include acne, adrenal insufficiency, allergies, alopecia, anemia, anorexia, anxiety, arthritis (osteo & rheumatoid), autism, cancer, chills, cystic fibrosis, depression, diabetes, digestive disorders, dry mouth, dysinsulinism, estrogen dominance, fatigue, fears, fractures, fungus, heart attack, high blood pressure, high cholesterol, Hodgkin's disease, hyperactivity, hypertension, hyperthyroid, low hydrochloric acid, hypoglycemia, infections, inflammation, insomnia, iron loss, jaundice, kidney disorders, libido decreased, lymphoma, mental illness, migraines, mood swings, multiple sclerosis, myocardial infarction, nausea, nervousness, osteoporosis, pancreatic dysfunction, panic attacks, paranoia, phobias, PMS, schizophrenia, senility, sexual dysfunction, spacey feeling, stuttering, stroke, tooth decay, toxemia of pregnancy, urinary tract infections, and yeast infections.

Lead can be found in such varied items as ash, auto exhaust, battery manufacturing, bone meal, canned fruit and juice, car batteries, cigarette smoke, coal combustion, colored inks, congenital intoxication, cosmetics, eating utensils, electroplating, household dust, glass production, hair dyes, industrial emissions, lead pipes, lead-glazed earthenware pottery, liver, mascara, metal polish, milk, newsprint, organ meats, paint, pencils, pesticides, produce near roads, putty, rain water, pvc containers, refineries, smelters, snow, tin cans with lead solder sealing (such as juices, vegetables), tobacco, toothpaste, toys, water (city/well), and wine. The effects include abdominal pain, adrenal insufficiency, allergies, anemia, anorexia, anxiety, arthritis (rheumatoid and osteo), attention deficit disorder, autism, back pain, behavioral disorders, blindness, cardiovascular disease, cartilage destruction, coordination loss, concentration loss, constipation, convulsions, deafness, depression, dyslexia, emotional instability, encephalitis, epilepsy, fatigue, gout, hallucinations, headaches, hostility, hyperactivity, hypertension, hypothyroid, impotence, immune suppression, decreased IQ, indigestion, infertility, insomnia, irritability, joint pain, kidney disorders, learning disability, liver dysfunction, loss of will, memory loss (long term), menstrual problems, mood swings, muscle aches, muscle weakness, muscular dystrophy, multiple sclerosis, myelopathy (spinal cord pathology), nausea, nephritis, nightmares, numbness, Parkinson's disease, peripheral neuropathies, psychosis, psychomotor dysfunction, pyorrhea, renal dysfunction, restlessness, retardation, schizophrenia, seizures, sterility, stillbirths, sudden infant death syndrome, tingling, tooth decay, vertigo, and unintentional weight loss.

In 1983, the U.S. Government began minting pennies made of zinc wafers coated in copper rather than out of pure copper. As it is not uncommon for animals to swallow pennies—hence, zinc toxicity became recognized. Other zinc sources include nuts, bolts, and zinc oxide based skin creams (such as diaper rash cream and sun screen). The clinical signs of zinc toxicosis include vomiting, diarrhea, red urine icterus (yellow mucous membranes), liver failure, kidney failure, and anemia. How zinc is able to produce hemolysis is not known.

B. Hemochromatosis

Hemochromatosis is the most common form of iron overload disease. Primary hemochromatosis, also called hereditary hemochromatosis, is an inherited disease. Secondary hemochromatosis is caused by anemia, alcoholism, and other disorders. Juvenile hemochromatosis and neonatal hemochromatosis are two additional forms of the disease. Juvenile hemochromatosis leads to severe iron overload and liver and heart disease in adolescents and young adults between the ages of 15 and 30. The neonatal form causes rapid iron buildup in a baby's liver that can lead to death.

Hemochromatosis is associated with the increased absorption of iron from the diet followed by a build up of iron in the body's organs leading to tissue damage. Without treatment, the disease can cause the liver, heart, and pancreas to fail. Iron is an essential nutrient found in many foods. The greatest amount is found in red meat and iron-fortified breads and cereals. In the body, iron becomes part of hemoglobin, a molecule in the blood that transports oxygen from the lungs to all body tissues.

Healthy people usually absorb about 10 percent of the iron contained in the food they eat, which meets normal dietary requirements. People with hemochromatosis absorb up to 30 percent of iron. Over time, they absorb and retain between five to 20 times more iron than the body needs. Because the body has no natural way to rid itself of the excess iron, it is stored in body tissues, specifically the liver, heart, and pancreas.

Hereditary hemochromatosis is one of the most common genetic disorders in the United States. It most often affects Caucasians of Northern European descent, although other ethnic groups are also affected. About five people out of 1,000—0.5 percent—of the U.S. Caucasian population carry two copies of the hemochromatosis gene and are susceptible to developing the disease. One out of every 8 to 12 people is a carrier of one abnormal gene. Hemochromatosis is less common in African Americans, Asian Americans, Hispanics/Latinos, and American Indians. Although both men and women can inherit the gene defect, men are more likely than women to be diagnosed with hereditary hemochromatosis at a younger age. On average, men develop symptoms and are diagnosed between 30 to 50 years of age. For women, the average age of diagnosis is about 50.

Joint pain is the most common complaint of people with hemochromatosis. Other common symptoms include fatigue, lack of energy, abdominal pain, loss of sex drive, and heart problems. However, many people have no symptoms when they are diagnosed. If the disease is not detected and treated early, iron may accumulate in body tissues and eventually lead to serious problems such as arthritis, liver disease, including an enlarged liver, cirrhosis, cancer, and liver failure, damage to the pancreas, possibly causing diabetes, heart abnormalities, such as irregular heart rhythms or congestive heart failure, impotence, early menopause, abnormal pigmentation of the skin, making it look gray or bronze, thyroid deficiency, and damage to the adrenal glands. A thorough medical history, physical examination, and routine blood tests help rule out other conditions that could be causing the symptoms. This information often provides helpful clues, such as a family history of arthritis or unexplained liver disease. Blood tests can determine whether the amount of iron stored in the body is too high. The transferrin saturation test reveals how much iron is bound to the protein that carries iron in the blood. Transferrin saturation values higher than 45 percent are considered too high. The total iron binding capacity test measures how well blood can transport iron, and the serum ferritin test correlates with the level of iron in the liver. If either of these tests shows higher than normal levels of iron in the body, doctors can order a special blood test to detect the underlying genetic mutation, which will confirm the diagnosis. If the mutation is not present, hereditary hemochromatosis is not the reason for the iron buildup and the doctor will look for other causes. A liver biopsy may be needed, in which case a tiny piece of liver tissue is removed and examined with a microscope. The biopsy will show how much iron has accumulated in the liver and whether the liver is damaged.

Treatment is simple, inexpensive, and safe. The first step is to rid the body of excess iron. This process is called phlebotomy, which means removing blood the same way it is drawn from donors at blood banks. Based on the severity of the iron overload, a pint of blood will be taken once or twice a week for several months to a year, and occasionally longer. Blood ferritin levels will be tested periodically to monitor iron levels. The goal is to bring blood ferritin levels to the low end of normal and keep them there. Depending on the lab, that means 25 to 50 micrograms of ferritin per liter of serum. Once iron levels return to normal, maintenance therapy begins, which involves giving a pint of blood every 2 to 4 months for life. Some people may need phlebotomies more often. An annual blood ferritin test will help determine how often blood should be removed. Regular follow-up with a specialist is also necessary.

If treatment begins before organs are damaged, associated conditions—such as liver disease, heart disease, arthritis, and diabetes—can be prevented. The outlook for people who already have these conditions at diagnosis depends on the degree of organ damage. For example, treating hemochromatosis can stop the progression of liver disease in its early stages, which leads to a normal life expectancy. However, if cirrhosis, or scarring of the liver, has developed, the person's risk of developing liver cancer increases, even if iron stores are reduced to normal levels. People with hemochromatosis should not take iron or vitamin C supplements. And those who have liver damage should not consume alcoholic beverages or raw seafood because they may further damage the liver. Treatment cannot cure the conditions associated with established hemochromatosis, but it will help most of them improve. The main exception is arthritis, which does not improve even after excess iron is removed.

Screening for hemochromatosis—testing people who have no symptoms—is not a routine part of medical care or checkups. However, researchers and public health officials do have some suggestions. Siblings of people who have hemochromatosis should have their blood tested to see if they have the disease or are carriers. Parents, children, and other close relatives of people who have the disease should consider being tested. Doctors should consider testing people who have joint disease, severe and continuing fatigue, heart disease, elevated liver enzymes, impotence, and diabetes because these conditions may result from hemochromatosis.

C. Wilson's Disease

Wilson's Disease causes the body to retain copper. The liver of a person who has Wilson's Disease does not release copper into bile as it should. Bile is a liquid produced by the liver that helps with digestion. As the intestines absorb copper from food, the copper builds up in the liver and injures liver tissue. Eventually, the damage causes the liver to release the copper directly into the bloodstream, which carries the copper throughout the body. The copper buildup leads to damage in the kidneys, brain, and eyes. If not treated, Wilson's disease can cause severe brain damage, liver failure, and death.

Wilson's Disease is hereditary. Symptoms usually appear between the ages of 6 and 20 years, but can begin as late as age 40. The most characteristic sign is the Kayser-Fleischer ring—a rusty brown ring around the cornea of the eye that can be seen only through an eye exam. Other signs depend on whether the damage occurs in the liver, blood, central nervous system, urinary system, or musculoskeletal system. Many signs can be detected only by a doctor, like swelling of the liver and spleen; fluid buildup in the lining of the abdomen; anemia; low platelet and white blood cell count in the blood; high levels of amino acids, protein, uric acid, and carbohydrates in urine; and softening of the bones. Some symptoms are more obvious, like jaundice, which appears as yellowing of the eyes and skin; vomiting blood; speech and language problems; tremors in the arms and hands; and rigid muscles.

Wilson's Disease is diagnosed through tests that measure the amount of copper in the blood, urine, and liver. An eye exam would detect the Kayser-Fleischer ring. The disease is treated with lifelong use of D-penicillamine or trientine hydrochloride, drugs that help remove copper from tissue, or zinc acetate, which stops the intestines from absorbing copper and promotes copper excretion. Patients will also need to take vitamin B₆ and follow a low-copper diet, which means avoiding mushrooms, nuts, chocolate, dried fruit, liver, and shellfish. Wilson's Disease requires lifelong treatment. If the disorder is detected early and treated correctly, a person with Wilson's Disease can enjoy completely normal health.

D. Infection

A variety of infectious agents can induce changes in the metal content of plasma and plasma proteins.

Fungal Diseases. Fungal diseases are caused by fungal and other mycotic pathogens (some of which are described in Human Mycoses (Beneke, 1979); Opportunistic Mycoses of Man and Other Animals (Smith, 1989); and Scripp's Antifungal Report, 1992); fungal diseases range from mycoses involving skin, hair, or mucous membranes, such as, but not limited to, Aspergillosis, Black piedra, Candidiasis, Chromomycosis, Cryptococcosis, Onychomycosis, or Otitis externa (otomycosis), Phaeohyphomycosis, Phycomycosis, Pityriasis versicolor, ringworm, Tinea barbae, Tinea capitis, Tinea corporis, Tinea cruris, Tinea favosa, Tinea imbricata, Tinea manuum, Tinea nigra (palmaris), Tinea pedis, Tinea unguium, Torulopsosis, Trichomycosis axillaris, White piedra, and their synonyms, to severe systemic or opportunistic infections, such as, but not limited to, Actinomycosis, Aspergillosis, Candidiasis, Chromomycosis, Coccidioidomycosis, Cryptococcosis, Entomophthoramycosis, Geotrichosis, Histoplasmosis, Mucormycosis, Mycetoma, Nocardiosis, North American Blastomycosis, Paracoccidioidomycosis, Phaeohyphomycosis, Phycomycosis, pneumocystic pneumonia, Pythiosis, Sporotrichosis, and Torulopsosis, and their synonyms, some of which may be fatal.

Known fungal and mycotic pathogens include, but are not limited to, Absidia spp., Actinomadura madurae, Actinomyces spp., Allescheria boydii, Alternaria spp., Anthopsis deltoidea, Apophysomyces elegans, Arnium leoporinum, Aspergillus spp., Aureobasidium pullulans, Basidiobolus ranarum, Bipolaris spp., Blastomyces dermatitidis, Candida spp., Cephalosporium spp., Chaetoconidium spp., Chaetomium spp., Cladosporium spp., Coccidioides immitis, Conidiobolus spp., Corynebacterium tenuis, Cryptococcus spp., Cunninghamella bertholletiae, Curvularia spp., Dactylaria spp., Epidermophyton spp., Epidermophyton fioccosum, Exserophilum spp., Exophiala spp., Fonsecaea spp., Fusarium spp., Geotrichum spp., Helminthosporium spp., Histoplasma spp., Lecythophora spp., Madurella spp., Malassezia furfur, Microsporum spp., Mucor spp., Mycocentrospora acerina, Nocardia spp., Paracoccidioides brasiliensis, Penicillium spp., Phaeosclera dematioides, Phaeoannellomyces spp., Phialemonium obovatum, Phialophora spp., Phoma spp., Piedraia hortai, Pneumocystis carinii, Pythium insidiosum, Rhinocladiella aquaspersa, Rhizomucor pusillus, Rhizopus spp., Saksenaea vasiformis, Sarcinomyces phaeomuriformis, Sporothrix schenckii, Syncephalastrum racemosum, Taeniolella boppii, Torulopsosis spp., Trichophyton spp., Trichosporon spp., Ulocladium chartarum, Wangiella dermatitidis, Xylohypha spp., Zygomyetes spp. and their synonyms. Other fungi that have pathogenic potential include, but are not limited to, Thermomucor indicae-seudaticae, Radiomyces spp., and other species of known pathogenic genera. These fungal organisms are ubiquitous in air, soil, food, decaying food, etc. Histoplasmoses, Blastomyces, and Coccidioides, for example, cause lower respiratory infections. Trichophyton rubrum causes difficult to eradicate nail infections. In some of the patients suffering with these diseases, the infection can become systemic causing fungal septicemia, or brain/meningal infection, leading to seizures and even death.

Viral Diseases. Viral diseases include, but are not limited to influenza A, B and C, parainfluenza (including types 1, 2, 3, and 4), paramyxoviruses, Newcastle disease virus, measles, mumps, adenoviruses, adenoassociated viruses, parvoviruses, Epstein-Barr virus, rhinoviruses, coxsackieviruses, echoviruses, reoviruses, rhabdoviruses, lymphocytic choriomeningitis, noroviruses, coronavirus, polioviruses, herpes simplex, human immunodeficiency viruses, cytomegaloviruses, papillomaviruses, virus B, varicella-zoster, poxviruses, rubella, rabies, picornaviruses, rotavirus, Kaposi associated herpes virus, herpes viruses type 1 and 2, hepatitis (including types A, B, and C), and respiratory syncytial virus (including types A and B).

Bacterial Diseases. Bacterial diseases include, but are not limited to, infection by the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like.

Protozoan Diseases. Protozoan or macroscopic diseases include infection by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

### VIII. Examples

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials

Blue dextran, phosphate buffered saline (PBS; 0.01 M phosphate, 2.7 mM KCl, 0.137 M NaCl) tablets, lysozyme (from chicken egg white), heparin (sodium salt) and a BCA protein determination kit were purchased from Sigma-Aldrich (St. Louis, Mo., USA), bovine serum albumin (BSA) from Amersham Pharmacia Biosciences (Buckinghamshire, UK) and plasma pure HNO₃ (67-70%) or HCl (36%) from SCP Science (Baie D'Urfe, QC, Canada). All solutions, including the mobile phase, were prepared with water from a Simplicity water purification system (Millipore, Billerica, Mass., USA).

### Example 2

### Analysis of Rabbit Plasma and Serum

Blood (˜7.0 ml) was collected from 4.5 h fasted male New Zealand white rabbits and the prepared plasma/serum was analyzed by SEC-ICP-AES within 30 min after blood collection. A schematic of the instrumental setup is presented in FIG. 1. A prepacked Superdex™ 200 GL 10/300 column (30×1.0 cm I.D., 13 μm particles, GE Healthcare, Bio-Sciences AB, Uppsala, Sweden) was used in conjunction with a Rheodyne 9010 PEEK injection valve (Rheodyne, Rhonert Park, Calif., USA) equipped with a 0.5 ml PEEK injection loop. PBS buffer of pH 7.4 (10 mM phosphate, 2.7 mM KCl and 137 mM NaCl) was prepared by dissolving PBS tablets in the appropriate volume of water (followed by pH adjustment if necessary) and filtration through 0.45 μm Nylon filter membranes (Mandel Scientific Company Inc., Guelph, ON, Canada). The flow-rate of the mobile phase throughout the chromatographic separation was maintained at 1.0 ml/min with a Waters 510 HPLC pump equipped with pharmaceutical grade polypropylene tubing (Mandel Scientific Company Inc., Guelph, ON, Canada). The packed and equilibrated column (50 ml mobile phase) was first injected with a mixture of BSA and lysozyme (1.2 mg and 0.62 mg in 0.5 ml) and the proteins were detected in the column effluent by on-line monitoring of the carbon emission line by ICP-AES at 193.091 nm. The peak shape of the lysozyme peak was used to calculate the number of theoretical plates (N) of the packed column and provided a qualitative measure of the column packing (N ˜23,000). More than 30 injections of plasma/serum can be performed with one column without any loss of chromatographic peak resolution. All separations were carried out at room temperature (22° C.).

The column exit of the SEC column was connected to the Meinhard concentric glass tube nebulizer of the ICP-AES with FEP Teflon tubing (54 cm, I.D. 0.5 mm). Simultaneous multielement-specific detection of C (193.091 nm), S (180.731 nm), Zn (213.856 nm), Fe (259.940 nm), Cu (324.754 and 224.700 nm) and P (213.618 nm) in the column effluent was achieved with a Prodigy, high-dispersion, radial-view ICP-AES (Teledyne Leeman Labs, Hudson, N.H., USA) at an Ar gas-flow rate of 19 L/min, an RF power of 1.3 kW and a nebulizer gas pressure of 35 psi. Time scans were performed using the time-resolved analysis mode (Salsa software version 3.0) and a data acquisition rate of 1 data point per 2 s. The raw data were imported into Sigmaplot 10 and smoothened using the bisquare algorithm. According to the void volume of the packed SEC column (determined by blue dextran), a 7.0 min delay was implemented between the injection and the beginning of data acquisition using a 1000 s data acquisition window. A representative simultaneous Cu, Fe and Zn-specific chromatogram of fresh rabbit plasma is shown in FIG. 2.

In order to investigate if freezing will affect the analytical results, a control experiment was conducted in which two aliquots of rabbit plasma samples were analyzed. The first plasma aliquot was directly analyzed (within 30 min of blood collection), whereas the second one was analyzed after freezing it for 6 days at −30° C. (n=5). The obtained simultaneous Cu, Fe and Zn-specific chromatograms were similar, which indicates that freezing did not alter the analytical results (data not shown).

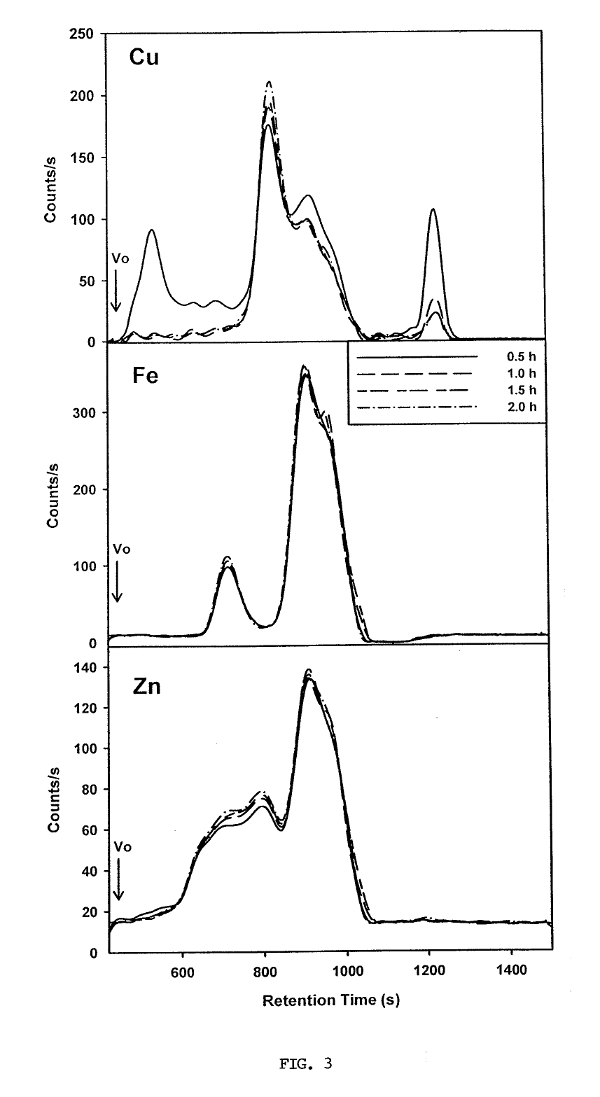

In addition, we investigated if ageing of the plasma affects the analytical results. Rabbit plasma was therefore analyzed by SEC-ICP-AES in 30 min intervals for a period of two hours. The results (FIG. 3) demonstrated that the Fe and Zn-specific chromatograms remained unchanged, whereas some changes were observed for Cu-proteins. These results also demonstrate that the developed analytical SEC-ICP-AES procedure produced results that are highly reproducible.

### Example 3

### Analysis of Human Plasma

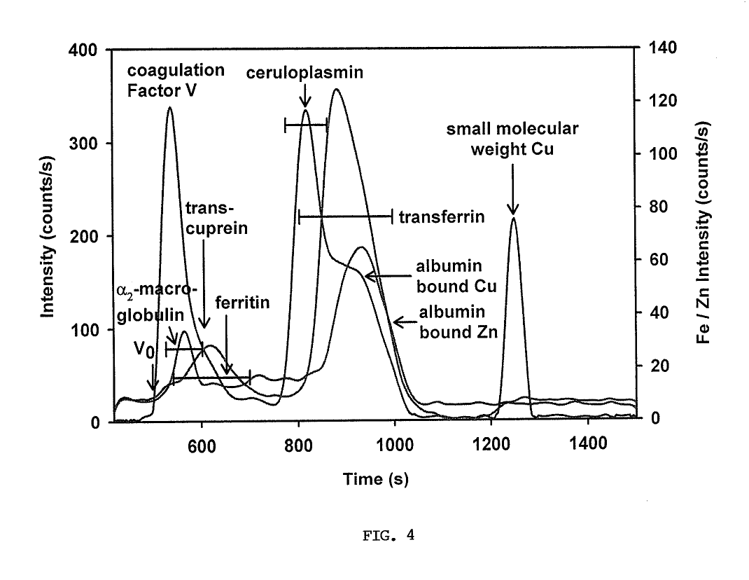

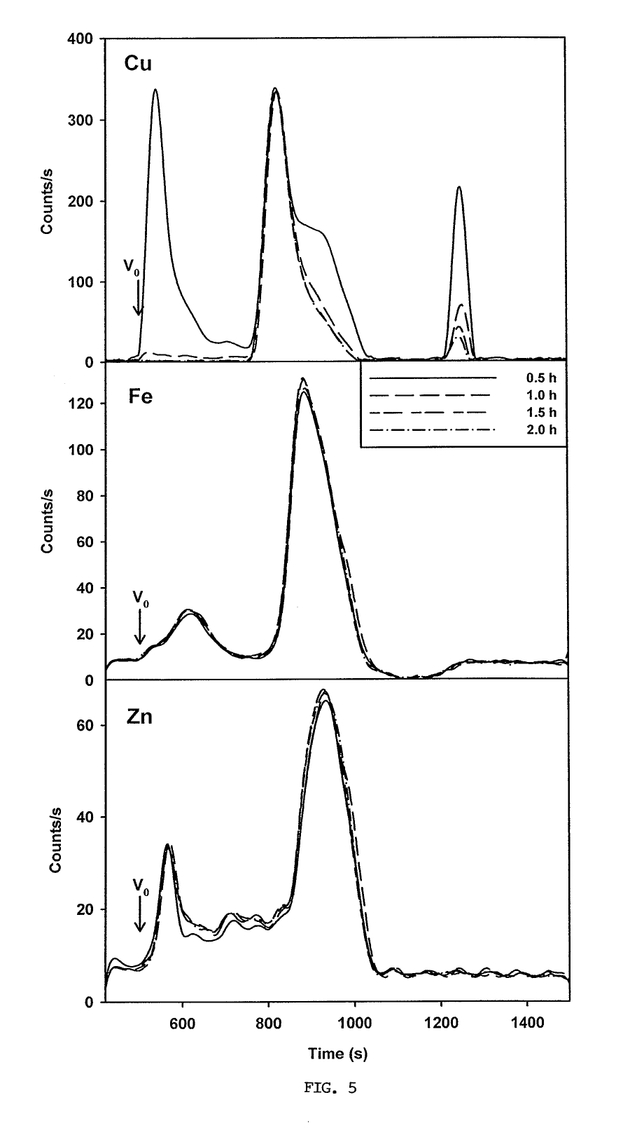

Blood (˜7.0 ml) was collected from healthy humans (after overnight fasting) and from hemochromatosis patients (non-fasted). The prepared plasma was analyzed by SEC-ICP-AES within 30 min after blood collection from healthy humans and as soon as logistically possible from the hemochromatosis patients. The SEC-ICP-AES analysis protocol was identical to that for rabbit plasma (see above). A representative simultaneous Cu, Fe and Zn-specific chromatogram for healthy human plasma is shown in FIG. 4. In addition, FIG. 5 displays the individual Cu, Fe and Zn-specific chromatograms obtained from the analysis of plasma from a healthy human over a 2 h period (30 min intervals). These results were essentially identical to those obtained for the time dependent analysis of rabbit plasma (FIG. 3). A comparison of the results that were obtained for healthy (n=9) and hemochromatosis patients (n=5) is provided in Table 3 (even though only a limited amount of patient plasma has been analyzed, differences between healthy and diseased subjects are apparent).

TABLE 3
COMPARISON OF PLASMA METALLPROTEIN CONENT
IN HEALTHLY AND HEMOCHROMATOSIS PATIENTS

| | | Control Group Average | Test Group Average |
|---|---|---|---|
| | | Metal Concentration | Metal Concentration |
| | | (μg/mL) ± 95% | (μg/mL) ± 95% |
| | | Confidence Interval; | Confidence Interval; |
| Element | Protein(s) | (N) | (N) |
| Cu | Peaks 1 & 2 | 0.349 ± 0.13 (9) | 0.500 ± 0.29 (5) |
| | Ceruloplasmin | 0.823 ± 0.13 (9) | 0.686 ± 0.030 (5) |
| | Albumin | 0.777 ± 0.099 (7) | 1.010 ± 0.14 (4) |
| | Small MW | 0.175 ± 0.061 (7) | 0.261 ± 0.12 (4) |
| Fe | Ferritin | 0.220 ± 0.10 (9) | 0.239 ± 0.080 (5) |
| | Transferrin | 1.110 ± 0.29 (7) | 1.662 ± 0.41 (4) |
| Zn | α₂₋ | 0.095 ± 0.015 (9) | 0.156 ± 0.016 (5) |
| | macroglobulin | | |
| | Peaks 2-4 | 0.202 ± 0.025 (9) | 0.233 ± 0.094 (5) |
| | Albumin | 0.710 ± 0.066 (9) | 0.683 ± 0.13 (4) |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. References

The following references, to the extent that they provide exemplary procedural or other details supplementary to those set forth herein, are specifically incorporated herein by reference.
U.S. Pat. No. 3,703,591
U.S. Pat. No. 3,718,433
U.S. Pat. No. 3,899,297
U.S. Pat. No. 3,907,645
U.S. Pat. No. 3,925,162
U.S. Pat. No. 3,925,164
U.S. Pat. No. 4,102,646
U.S. Pat. No. 4,425,427
U.S. Pat. No. 6,248,592
Banauch et al., Z. Klin, Chem. Klin, Biochem., 13:101-107, 1975.
Beneke, In: Human Mycoses, Upjohn Co., Kalamazoo, Mich., 1979.
Chem. Abstract 88, 83042, 1978.
Chem. Analy. (Warsaw), 22(1):27-35, 1977.
Craig, W. Y., Ledue, T. B., and Ritchie, R. F., in Plasma Proteins: Clinical Utility and Interpretation. Newark, Dade Behring, Inc, 2000.
Kageyama, Clinica Chemica Acta, 31:421-426, 1971.
Luderer, In: An Automated Method for the Enzymatic Determination of Triglycerides in Serum, Wiley-Inter Science, N.Y., 1975.
Scrip's Antifungal Report, PJB Publications Ltd., 1992.
Smith, In: Opportunistic Mycoses of Man and Other Animals, CAB International: Wallingford, UK, 1989.
Sterns, In: The Practice of Absorption Spectro-Photometry, Wiley-Inter Science, N.Y., 1969.
West German Auslegeschrift 2558536

What is claimed is:

1. A method of measuring metal distribution in plasma or serum comprising:
(a) providing a plasma or serum sample;
(b) subjecting said plasma or serum sample to size exclusion chromatography (SEC) to obtain SEC effluent comprising separated plasma or serum proteins;
(c) feeding said SEC effluent obtained in step (b) directly into an inductively-coupled plasma atomic emission spectrometer (ICP-AES) to determine the metal content thereof; and
(d) associating the metal content determined in step (c) with plasma or serum proteins separated in step (b).

2. The method of claim 1, wherein the time from step (b) to step (d) is less than 30 minutes.

3. The method of claim 1, wherein said metal is selected from Cu, Zn, and Fe.

4. The method of claim 1, wherein said AES is inductively coupled plasma AES.

5. The method of claim 1, wherein said plasma or serum is from rabbit, dog, cat, rat, mouse, sheep, goat, cow, pig or horse.

6. The method of claim 1, wherein said plasma or serum is from a human.

7. The method of claim 1, wherein said human plasma or serum is obtained from a subject that is suspected of having a condition that effects metalloprotein content of blood.

8. The method of claim 7, wherein said condition is hemochromatosis, Wilson's Disease, metal poisoning or an infection.

9. The method of claim 1, further comprising the step of obtaining said blood sample from a subject and preparing said plasma or serum sample therefrom.

10. The method of claim 1, wherein the amount of the plasma or serum sample subjected to SEC is 500 μl.

11. The method of claim 1, wherein step (d) comprises computer-assisted processing of data from said SEC-ICP-AES.

12. The method of claim 7, further comprising assessing the metal content of said human subject from at least two different time points.

13. The method of claim 1, wherein the plasma or serum sample is essentially free of red blood cells and hemoglobin from lysed red blood cells.
